(19) United States
(12) Patent Application Publication
(10) Pub. No.: US 2017/0240444 A1
(43) Pub. Date: Aug. 24, 2017

(54) BIOPELLET REACTOR WITH CYCLONIC FLUIDIZING PLATE

(71) Applicant: Thomas Blaha, Chardon, OH (US)

(72) Inventor: Thomas Blaha, Chardon, OH (US)

(21) Appl. No.: 15/046,474

(22) Filed: Feb. 18, 2016

(51) Int. Cl. C02F 3/08; C12M 1/02; A01K 63/04; C12M 1/12

(52) U.S. Cl. CPC C02F 3/085; C12M 25/20; C12M 27/00; C12M 25/06; A01K 63/04; C02F 2103/20

(57) ABSTRACT

A biopellet reactor for marine aquariums. The invention is functionally dependent upon a unique fluidizing plate located between the water inlet port at the base and the media reaction chamber above. This fluidizing plate converts the upward momentum of the incoming water stream into a cyclonic flow in the reaction chamber. This cyclonic flow completely suspends and fluidizes the biopellets within the reaction chamber

### CROSS-REFERENCE TO RELATED APPLICATIONS

This Application claims the benefit of U.S. Provisional Patent Application No. 62/118,609, filed Feb. 20, 2015, the entire contents of which is incorporated herein by reference.

### BACKGROUND OF THE INVENTION

1. Field of the Invention

This invention relates to the field of filtration equipment for Marine Aquariums. More specifically, the invention comprises of a biopellet reactor intended to remove or reduce nitrates and phosphates from marine aquarium water.

2. Description of the Related Art

There currently several biopellet reactor designs available. The primary function of a biopellet reactor is mix tank water with bio-degradable plastic pellets. Naturally occurring bacteria in the aquarium water utilize the biodegradable plastic pellets as a carbon source for metabolism allowing them to grow and multiply on the surface of the pellets. In addition to utilizing the pellets as a carbon source, the bacteria also draw phosphate and nitrate from the aquarium water as sources of phosphorus and nitrogen which are necessary for cell growth and replication. Nitrate and phosphate are also used by algae and other nuisance aquarium inhabitants so biopellet reactors have been shown to improve overall aquarium water quality and reduce the growth of nuisance organisms.

A standard biopellet reactor provides several functions in an aquarium filtration system. First, it provides a retaining system that allows tank water to pass through the pellets without allowing the pellets to escape into the main tank. Second, it allows water from the main tank to pass through the pellets, delivering available nitrate and phosphate to the bacteria growing the pellets. Additionally, it allows a return path for that water to reenter the main tank. Finally, it provides enough water flow through the pellets to keep them suspended or “Fluidized” within the water being treated. During normal function, the desired bacterial growth on the pellet surface creates a bacterial film. That film is sticky and without proper flow and motion, the pellets will stick to each other and clump up. Clumped pellets and lack of proper flow can lead to the generation of compounds such as sulphur dioxided which are detrimental to the aquarium. The water passing through the pellets is also sufficiently strong as to strip some of the bacterial film from the pellets. This stripped film exits the reactor where the bacteria can then be extracted from the water with another filter such as a protein skimmer, or they may be consumed by other tank inhabitants such as corals. By removing the bacteria that consumed the phosphate and nitrate within the tank water, those compounds are effectively eliminated from the tank

Current designs in use fluidize the pellets by utilizing an electric water pump to pump tank water at high velocity through the pellets inside a chamber separated from the tank water by the pump itself on one end and a screen on the other with openings sufficiently small as to prevent the vast majority of the pellets from escaping the reaction chamber. Water passing through the screen then reenters the main tank system. The biopellets are denser then water so they naturally sink to the the bottom of the chamber. In existing systems, the fluidizing is accomplished by directing the flow of the pumped water in the chamber into, or through the biopellets resting in the bottom of the reaction chamber. These devices direct the water from the top down, with the water stream deflecting off the bottom of the reactor causing up currents which fluidize the pellets, or from the bottom up through small holes which direct water current straight upward, lifting the pellets with them. Alternatively, fluidization has also been achieve with a combination of the above with the mechanical mixing of the pellets by a water flow driven stirring device. These methods of fluidization do not provide uniform or consistent suspension of the pellets within the system, or they rely on moving (mechanical) parts to do so. The present invention utilizes no moving parts and provides uniform fluidization of the pellets within the reactor.

### BRIEF SUMMARY OF THE PRESENT INVENTION

The present invention comprises a biopellet reactor with a unique method of pellet fluidization. The reactor is setup and operated in the same way as existing reactors. It consists of an inlet pipe which directs water from an external water pump into the base of the reactor. The water is forced through a cyclonic fluidizing plate into the reaction chamber from below the pellets. The unique shape and configuration of the fluidizing plate creates upward pressure and a cyclonic effect on the water and pellets above it, causing the pellets to be fluidized in a uniform and controllable fashion. At the top of the reactor is screen plate that allows the water that has passed through the reactor to return to the tank through a pipe, while retaining the biopellets in the reaction chamber.

### BRIEF DESCRIPTION OF THE SEVERAL VIEWS OF THE DRAWINGS

FIG. 1 shows a perspective drawing of an assembled unit

FIG. 2 shows an exploded view of a complete unit

FIG. 3 shows a perspective and side view of the fluidizing plate

FIG. 4 shows a top and bottom view of the fluidizing plate

REFERENCE NUMERALS IN THE DRAWINGS

| | | | |
|---|---|---|---|
| 1. | Biopellet Reactor | 2. | Reactor Base |
| 3. | Fluidizing Plate | 4. | Reaction Chamber |
| 5. | Exit Strainer | 6. | Water inlet |
| 7. | Water Outlet | 8. | Nylon Screws |
| 9. | O-Ring | 10. | Top Mounting Plate |
| 11. | Water Passthtough | 12. | Water Pass through |
| 13. | Inlet Outlet offset | 14. | Deflection face angle |
| 15. | Deflection face | 16. | Fluid path entry |
| 17. | Angle between Fluid Path Exits | 18. | Fluid Path Curvature |
| 19. | Fluid Path Distance from outside | 20. | Fluid Path Width |

### DETAILED DESCRIPTION OF THE INVENTION

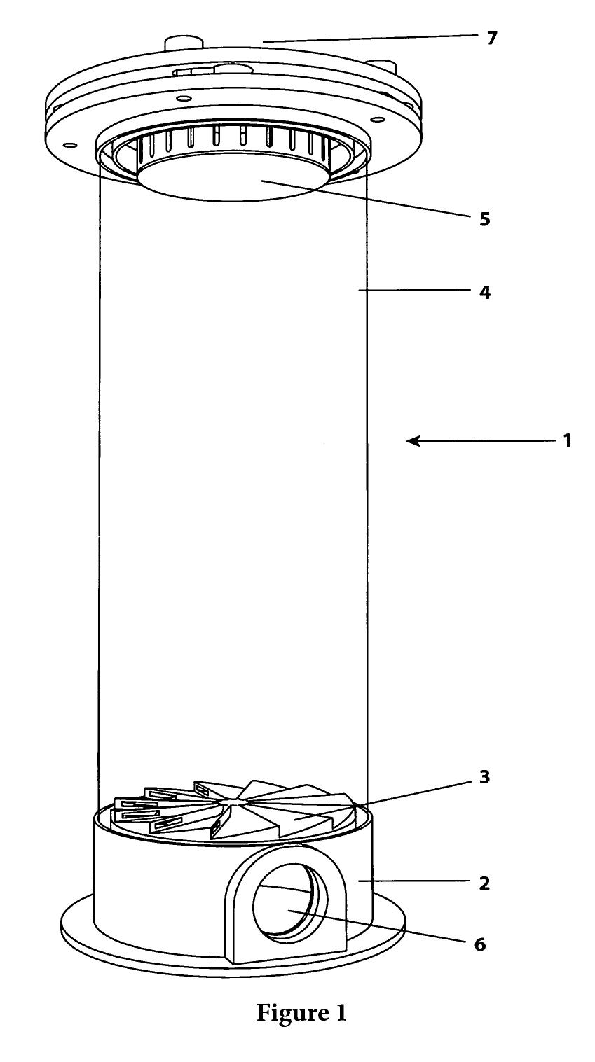

FIG. 1 shows a perspective view of the present invention in an assembled state. Biopellet Reactor 1 includes the Reactor Base 2 where water enters the reactor through water inlet 6. Water is pushed through water inlet 6 via an external water pump, which is not shown and not part of the invention. Fluidizing plate 3 is affixed and sealed to the top of the reactor base 2. Fluidizing plate 3 contains a series of holes which allow water to pass from the reactor base into the Reaction Chamber 4. The fluidizing plate prevents the biopellets contained in the reaction chamber from entering the reactor base chamber. It is in the reaction chamber that the biopellets mix with the incoming, nutrient rich aquarium water, allowing bacterial growth to occur thus lowering nutrient levels in the water. At the top of the reaction chamber, is the exit strainer 5 which allows the water to pass out of the reactor while retaining the biopellets within the reaction chamber. The water then passes through the water outlet 7.

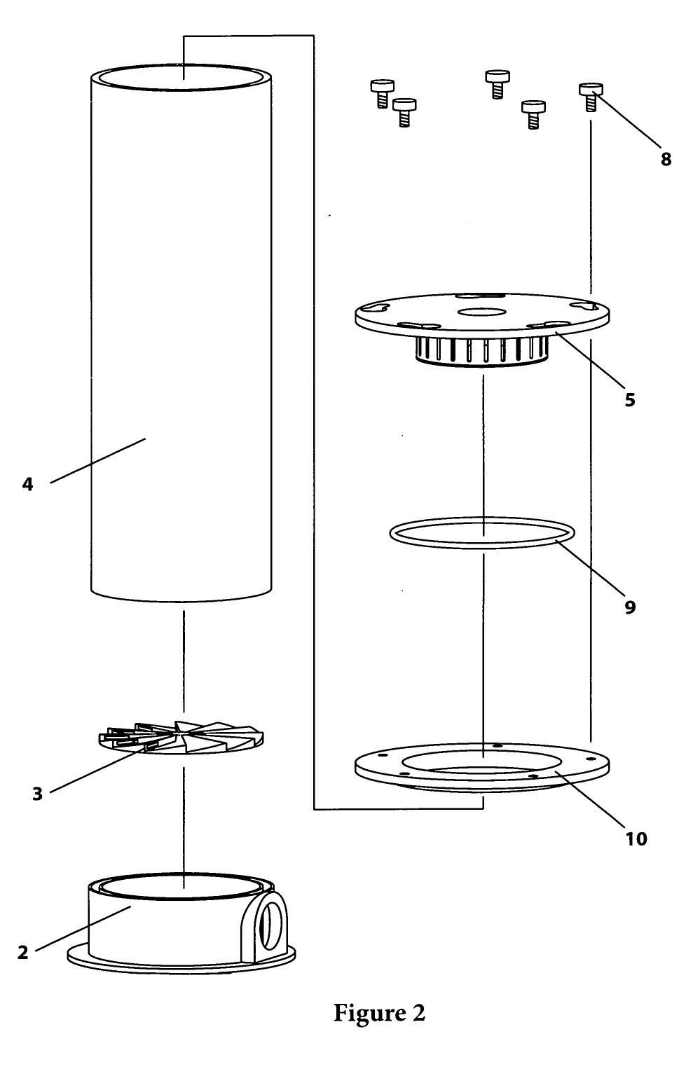

FIG. 2 show an exploded view detailing how the units components fit together to make the complete unit. Nylon screws 8 are used to attach the top exit strainer 5 to the top mounting plate 10. An O-Ring 9 is compressed between the exit strainer and the top mounting plate to prevent water leakage from the assembled unit. The top mounting plate is glued to the top of the reaction chamber 4. The fluidizing plate 3 is sealed to the reactor base with glue or other mechanism such as a nylon screw. The reaction chamber is then glued to the base such that the fluidizing plate is located inside the reaction chamber tube. This is the assembly of large units. Small units have the same basic components, but the nylon screws are replaced with a female threaded retainer cap and the top mounting plate is replaced with a male threaded mounting plate. The retainer cap threads directly onto the mounting plate compressing the retaining screen and o-ring against the mounting plate.

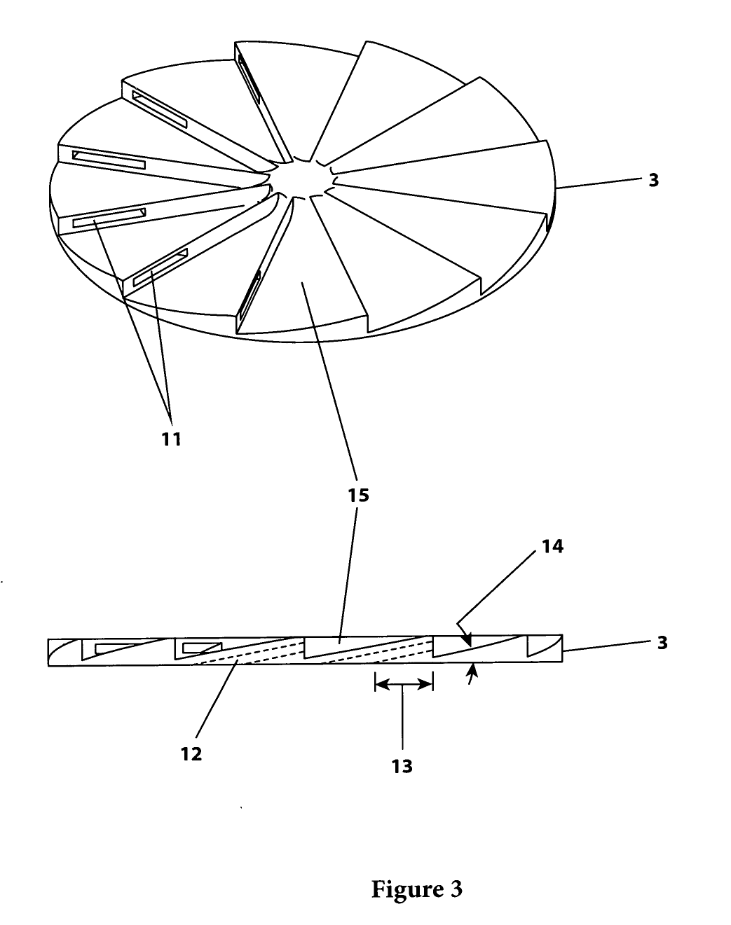

FIG. 3 shows an isometric and side view of the fluidizing plate 3. The fluidizing plate includes a series of rectangular water pass through holes 11 that are located near the outside diameter of the plate. The pass through hole path 12 is shown in the side view as hidden dotted lines. The distance between the bottom inlet and top outlet 13 is sufficient that the pass through hole is angled through the plate at about the same angle as the deflection face angle 14. The distance can very depending on the diameter of the fluidizing plate, but must be greater then 0. The outlet void zone 15 is the space between the outlet hole face and the top surface of the adjacent deflection face.

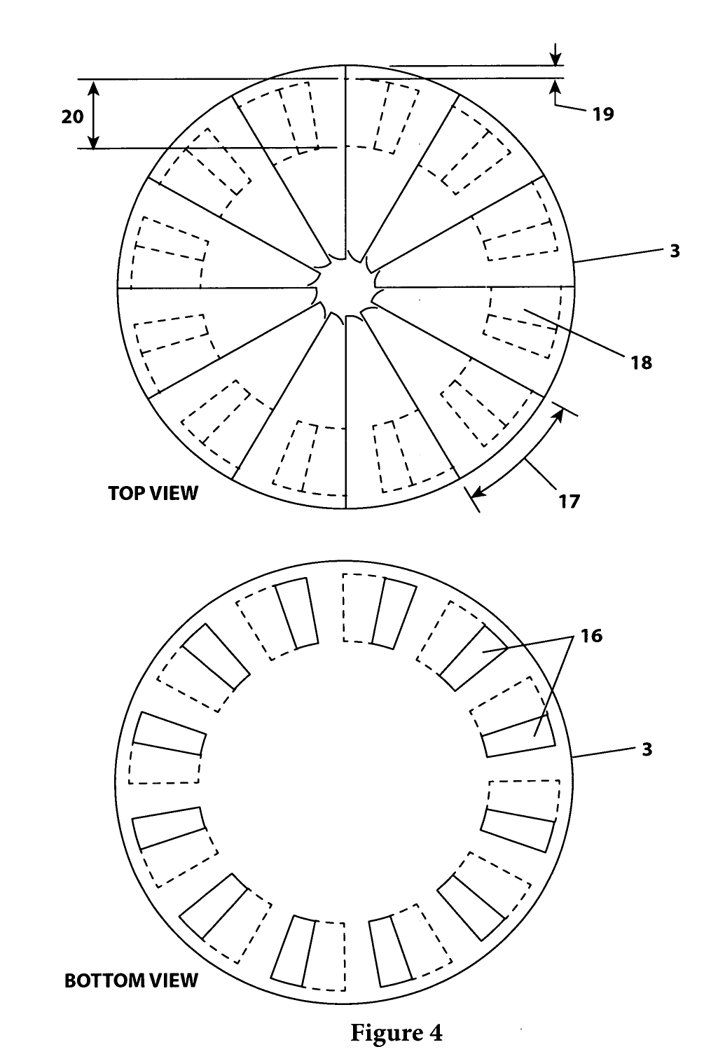

FIG. 4 shows top and bottom views of the fluidizing plate 3. The opening to the pass through holes in the bottom of the plate 16 are shown. The pass through hole is a consistent size and passes straight through the plate at a consistent angle. Those two factors combined result in the pass through holes in the bottom of the plate are larger then the exit hole in the top of the plate. The angle between the top exit faces on the top of the plate 17 is the same for all adjacent faces around the plate. That angle can very depending the diameter of the plate, with smaller plates having a larger angle. The pass through holes are all the same height regardless of plate diameter, which necessitates that all plates be the same thickness. As the diameter if the plate decreases the circumference decreases as well. The pass through holes are radiused to be parallel 18 to the outside plate diameter and they become shorter as the circumference decreases. As the pass through holes get shorter the angle of the pass through hole becomes steeper (distance 13 decreases). To maintain a proper angle of the pass through hole, the angle between exit faces can be increased. For example, a 4″ diameter fluidizing plate will have an exit face angle of 30 degrees, whereas a 1″ diameter plate will have a 45 degree exit face angle.

The distance of the exit hole from the outside edge of the fluidizing plate 19 is constant regardless of plate diameter. The width of the exit hole 20 is dependent upon expected flow rate of the biopellet reactor. The height of the exit hole is fixed to a size small enough to prevent the biopellets from passing through to the base chamber if the pump is turned off. Acceptable width is such that the summation of the surface areas of the exit holes is large enough to allow the total flow rate of water passing through the plate to match or exceed the flow rate necessary for proper fluidization of the biopellets.

During operation, water passes through the fluidizing plate from bottom to top. It enters the plate with an upward vector and the pass through holes alter the direction of flow to be parallel to the top surface of the plate. The position of the exit holes in the top of the plate near the outside walls of the reactor force the water to flow in a circular motion around the reactor chamber as it rises toward the top of the reactor. The biopellets are lifted above the fluidizing plate by the pressure created by the flow of water over the top surface of the fluidizing plate. This creates a layer of water above the plate which completely suspends the mass of pellets. The cyclonic motion of that layer causes the biopellets above it to spin and mix around the chamber. This rotation generates centrifugal force on the pellets which forces them toward the walls of the chamber. Gravity pulls the pellets back down toward the fluidizing plate. The combination of the uplifting force generated but the fluidizing plate, centrifugal and gravitational forces balance out to confine the mass of biopellets into a fully suspended band which is spinning and mixing within the chamber. The use of an external gate or ball valve to control the input flow of water can control the size, location and motion within the mass of biopellets.

Although the preceding description of the invention contains many details, it should not be taken as limiting the scope of the invention but rather providing illustration of a present working model. For example, the diameter of the fluidizing plate/media reactor and the number or size of the outlet holes can very depending on the overall size of the reactor. Small diameter reactors require fewer holes and smaller holes in the fluidizing plate to achieve the same functionality. Such alterations would not materially alter the nature of the invention and would in fact be necessary to achieve proper function. Thus, the scope of the invention should be fixed by the following claims rather then and specific examples provided.

Having described my invention, I claim:

1. An aquarium biopellet reactor comprising
a. A bottom fed inlet chamber
b. A reaction chamber where the biopellets are fluidized with tank water.
b. A screening device at the top of the reaction chamber for biopellet retention in the chamber
c. A unique fluidizing plate between the bottom inlet and the reaction chamber

2. An aquarium biopellet reactor as recited in claim 1, wherein the fluidizing plate has the following features:
a. Multiple rectangular fluid paths which channel water from the reactor base horizontally across the top of the fluidizing plate.
b. Fluid paths cited in 2a distributed radially around the fluidizing plate at uniform angles
c. A top surface with angled faces between the fluid path exit openings to direct water flow upward.
d. Fluid paths cited in 2a which have bottom entry openings larger then the exit openings on the top side
e. Fluid paths cited in 2a which are sized vertically to prevent passage of biopellets back into the base chamber, and horizontally to govern maximum water flow through the reactor.
f. Fluid paths cited in 2a which curve parallel to the outside diameter of the plate while passing through the plate.

3. An aquarium biopellet reactor as recited in claim 1, wherein the fluidizing plate generates a cyclonic or vortex rotation of the water within the reaction chamber with no moving parts.

4. An aquarium biopellet reactor as recited in claim 1, wherein the fluidizing plate generates a pressure gradient between the biopellet mass and the fluidizing plate, causing the suspension of the entire biopellet mass above the plate.

5. An aquarium biopellet reactor as recited in claim 1, wherein the combination of gravitational force, the centrifugal forces induced by the cyclonic motion cited in claim 2, and the upward pressure force cited in claim 3 forces the biopellets to be fluidized in a uniform, consistent and controllable form within the reaction chamber.
